(19) United States
(12) Patent Application Publication
(10) Pub. No.: US 2020/0163859 A1
(43) Pub. Date: May 28, 2020

(54) Composition, an Article for Personal Care Comprising the Composition, a Method for Preparing the Article for Personal Care and use of the Article for Personal Care

(71) Applicant: Arion Holding B.V., Heerlen (NL)

(72) Inventors: Erik Joosten, Beek (NL); Jacobus Otte, Heerlen (NL)

(21) Appl. No.: 16/689,859

(22) Filed: Nov. 20, 2019

(30) Foreign Application Priority Data
Nov. 22, 2018 (NL) 2022049

(51) Int. Cl. A61K 8/73; A61K 8/25; A61K 8/81; A61K 8/34; A61K 8/891; A61K 8/02; A61K 31/728; A61K 47/10; A61K 47/34; A61K 47/32; A61K 47/02

(52) U.S. Cl. CPC A61K 8/735; A61K 8/25; A61K 8/8158; A61K 8/345; A61K 8/891; A61K 8/0208; A61K 2800/594; A61K 47/10; A61K 47/34; A61K 47/32; A61K 47/02; A61K 2800/524; A61K 31/728

(57) ABSTRACT

A composition is provided comprising at least a layered silicate, an acrylate polymer, a preservative compound, a humectant compound, a hyaluronic acid or a salt derivative thereof, and water. The composition may be used in a wipe for personal care. A method for preparing the wipe for personal care, as well as the use of the wipe for personal care is disclosed.

### CROSS-REFERENCE TO RELATED APPLICATIONS

This application claims the benefit of and priority to Netherlands application 2022049, filed Nov. 22, 2018, the contents of which are incorporated herein by reference.

### FIELD OF THE INVENTION

The invention relates to a composition that is preferably used in an article for personal care. The invention further relates to an article for personal care comprising the composition according to the present invention, and to a process for preparing the article for personal care. The invention is not limited to a specific article. Suitable articles may comprise a sheet or wipe, but other geometries may also be possible, such as a glove to cover a hand, or a cap to cover a user's hair.

### BACKGROUND

A wipe for personal care is generally known for cleaning and/or protecting a skin. The wipe comprises a nonwoven or textile carrier and a composition, such as a liquid or a thin lotion, for cleaning and/or protecting the skin. A disadvantage of the known composition is that it is a liquid having low viscosity, which, when wiping the cleaning wipe including the composition over the skin, has a relatively low substantivity to the skin only, and possibly also a low visibility on the skin.

Other than aqueous solutions, wet wipes may be impregnated with more substantial lotions, like light cream emulsions. Such creams are designed to impart more protective or caring characteristics, which may be attributed in part to their oil phase, and is typically associated with the use of fatty acid esters.

Viscosity (η) is a measure of a fluid's internal flow resistance. It may be defined as the resistance, which a fluid shows when being deformed, and it gives information on how thick a fluid is and how easily it flows.

The inclusion of functional additives and excipients, including active ingredients, emollients, and humectants and so on, usually results in a thicker lotion with higher viscosity. In prior art, such lotions would commonly be based on emulsion technology, for example oil-in-water or water-in-oil emulsion formulas. Typical emulsifiers would include esters of fatty acids such as stearic acid, lauric acid, palmitic acid, and myristic acid. These fatty acids could for example be esterified with glycerin or polyethylene glycols.

As cream emulsions usually have a considerably higher viscosity than aqueous solutions, traditional impregnation processes at room temperature may be less appropriate. Traditional emulsions require hot processing, and may then be used for impregnation while hot or during the cooling phase, to take advantage of reduced viscosity while hot to overcome impregnation difficulties, thus obtaining even impregnation of the nonwoven material of the wipe.

High viscosity in lotions results in critical technical challenges during pumping of the lotion through the piping system and the impregnation process. Viscous lotions are not easily pumped through the piping system, or cannot be pumped at all. Viscous lotions more easily clog the spray nozzles, and viscous lotions tend to have less favourable spreading characteristics, thus potentially resulting in uneven dispersion of the lotion in the nonwoven cloth. Elevated viscosity of lotions for impregnation of nonwovens is therefore not preferred.

Hot process manufacturing has higher manufacturing cost implications, and has a larger energy footprint than cold process manufacturing. Cold processing represents savings in time and energy, and dedicated equipment with heating and cooling facilities are not required. Cold process manufacturing of stable traditional cream emulsions, such as the stearates types, is unlikely.

Impregnation of nonwoven cloth materials usually involves extensive piping systems, with in-line impregnation nozzles at the end. The flow rate of the impregnation lotion through the nozzles has to be sufficiently high, to keep up with the velocity of the nonwoven substrate material. Both impregnation lotion and cloth material are selected such that the lotion is readily absorbed, evenly dispersed, and adequately retained by the nonwoven material.

Embodiments of the invention aim to provide a composition usable for personal care such as for cleaning and/or protecting a skin, wherein an article including the composition is easily prepared and wherein the composition attributes and/or enhances a cleaning and/or protection of the skin. Particular embodiments aim to provide a process for preparing the article, wherein the article including the composition is easily prepared at a lower temperature such as room temperature and wherein the composition attributes and/or enhances a cleaning and/or protection of the skin. Other particular embodiments aim to provide a composition, wherein the composition further attributes and/or enhances a moisturizing behavior of the skin, which has been cleaned by said composition.

### SUMMARY OF THE INVENTION

According to a first aspect of the invention there is provided a composition in accordance with claim 1. The composition comprises a layered silicate, an acrylate polymer, a preservative compound, a humectant compound, further a hyaluronic acid or a salt derivative thereof, and water.

The current invention has addressed the challenges, utilizing a novel balanced mixture of thickening ingredients: layered silicate and acrylate polymer. The composition is based on hydrogel technology, which suspends caring ingredients rather than emulsifying them. The thickening system of the composition has been designed to be shear thinning, thus allowing for adequate manufacturing pumping conditions and nonwoven impregnation upon nozzle-spraying.

The suspension of the layered silicate in combination with the acrylate polymer forms a hydrogel base, which provides a thickening effect and a shear thinning behavior to the composition. In particular, the layered silicate in the acrylate polymer forms a suspension system inside the composition. The composition is thickened in a rest state, when no shear is applied. During manufacturing of an article comprising the composition, the composition may easily be transported, e.g. by pumping, and applied into a textile carrier of the article due to its shear thinning behavior. In this way, the article including the composition is easily prepared.

During use of the article by wiping it over the skin, the composition forms a relatively abundant or rich layer on the skin, due to its relatively high viscosity during a wiping movement. In this way, the composition enhances a cleaning and/or protection of the skin.

In embodiments, the layered silicate may be a clay mineral and may be a synthetic layered silicate. Clay minerals are hydrous aluminum phyllosilicates. Preferably, the layered silicate is a phyllosilicate.

Layered silicate can be classified as 1:1 or 2:1, this originates because they are fundamentally built of tetrahedral silicate sheets and octahedral hydroxide sheets. A 1:1 layered silicate would consist of one tetrahedral sheet and one octahedral sheet, and examples would be kaolinite and serpentine. A 2:1 layered silicate consists of an octahedral sheet sandwiched between two tetrahedral sheets, and examples are talc, vermiculite and montmorillonite.

In an exemplary embodiment, the layered silicate is a smectite. The smectite group includes dioctahedral smectites such as montmorillonite, nontronite and beidellite and trioctahedral smectites, for example saponite.

In a particular embodiment, the smectite is selected from the group consisting of bentonite, hectorite, montmorillonite, nontronite, beidellite, saponite, stevensite and mixtures thereof. In a preferred embodiment, the smectite is a bentonite. Bentonite is an absorbent aluminum phyllosilicate layered silicate. In an example, the smectite is a magnesium aluminum silicate.

In another aspect of the present invention, a hydrogel suspension composition is provided containing a magnesium aluminum silicate and an acrylamidepolymer, such as polyacrylate crosspolymer-11.

In an exemplary embodiment, the amount of the layered silicate is at least 0.3 wt. %. preferably at least 0.5 wt. %. more preferably at least 0.8 wtt. %, based on the total weight of the composition. When referring to the total weight of the composition throughout the description, the weight of the water is included. Said amount enhances a control on the shear thinning behavior of the composition.

In an exemplary embodiment, the amount of the layered silicate is at most 5.0 wt. %. preferably at most 2.5 wt. %. more preferably at most 1.8 w-t.%, based on the total weight of the composition.

In an exemplary embodiment, the acrylate polymer comprises acrylamide units. The acrylamide units of the acrylate polymer enhance the control on the shear thinning behavior of the composition in combination with the selected layered silicate. In a particular embodiment, a part of the acrylamide units comprises a sulphonic acid group, which is modified by an ammonium salt. The sulphonic acid groups of the acrylate polymer, which are modified by an ammonium salt, further enhance the control on the shear thinning behavior of the composition in combination with the selected layered silicate. Applying a poly(vinyl-acrylate) copolymer to the composition is less preferred. More preferably, an embodiment of the composition does not comprise an acryloyldimethyltaurate copolymer, such as Aristoflex AVC (INCI: Ammonium Acryloyldimethyltaurate/VP Copolymer).

In an exemplary embodiment, the amount of the acrylate polymer is at most 1.0 wt. %. preferably at most 0.5 wt. %. based on the total weight of the composition. Said amount enhances a control on the shear thinning behavior of the composition.

In an exemplary embodiment, the amount of the acrylate polymer is at least 0.01 wt. %. preferably at least 0.1 wt. %. based on the total weight of the composition.

In an exemplary embodiment, the ratio between the layered silicate and the acrylate polymer based on their weight is in the range from 1.0:1.0 to 5.0:1.0, preferably from 2.6:1.0 to 4.3:1.0. The weight ratio between the layered silicate and the acrylate polymer enhances a formation of a relatively abundant layer of the composition on the skin.

According to the invention, the composition further comprises hyaluronic acid or a salt derivative thereof. The addition of hyaluronic acid or its salt has been shown to further strengthen the viscosity of the composition, without negatively affecting either yield point or thixotropy of the composition. The hyaluronic acid may be a natural glucosaminoglycan having a high molecular weight. The hyaluronic acid or a salt derivative thereof may also act as an effective moisturizer for the skin, which easily forms a film on the skin due to its high molecular weight. In this way, a moisturizing effect of the composition is further enhanced.

In an exemplary embodiment, the amount of said hyaluronic acid including the salt derivatives thereof is up to 0.5 wt. % based on the total weight of the composition. The hyaluronic acid and/or its salt derivatives preferably have a molecular weight of at least 800 kDa, more preferably of at least 1500 kDa.

In an exemplary embodiment, the salt derivative of the hyaluronic acid is selected from at least one of a sodium salt, a potassium salt, and a zinc salt. The salt derivative of the hyaluronic acid may suitably be selected to improve the moisturizing effect of the hyaluronic acid in the composition. In a particular embodiment, the salt derivative of the hyaluronic acid is a zinc salt thereof. The zinc salt derivative of the hyaluronic acid additionally supports skin recovering or skin healing.

In an exemplary embodiment, the composition has a dynamic viscosity exceeding 50 mPas, preferably exceeding 100 mPas. The viscosity, the yield point and the thixotropy of a composition are measured according to ISO 3219. The measurement is carried out at 23.0° C. Compositions with a dynamic viscosity up to typically 50 or 100 mPas can be pumped through the piping system and nozzles without any specific problems. In case of a dynamic viscosity exceeding typically 100 mPas, thixotropy may be needed to enable processing the lotion in the piping and nozzle system.

In an exemplary embodiment, the composition has a yield point between 25 and 250 Pa, preferably between 50 and 150 Pa. The viscosity, the yield point and the thixotropy of a composition are measured according to ISO 3219. The measurement is carried out at 23.0° C. Preferably, the measured yield point exceeds 50 Pa.

In an exemplary embodiment, the composition has a thixotropy of 100 to 1000 Pa/s, preferably between 250 and 750 Pa/s. The viscosity, the yield point and the thixotropy of a composition are measured according to ISO 3219. The measurement is carried out at 23.0° C. Preferably, the thixotropy is around 500 Pa/s.

In an exemplary embodiment, the humectant compound comprises at least one of the group consisting of an alkyl glycol, such as propylene glycol, dipropylene glycol, butylene glycol, pentylene glycol, etc., glycerin, and glycerin derivatives. The humectant compound attributes to or enhances a moisturizing effect of the skin.

In an exemplary embodiment, the composition additionally comprises a poly(dimethyl)siloxane polymer. The poly(dimethyl)siloxane polymer has a low surface tension. The poly(dimethyl)siloxane polymer is substantially immiscible to water. The poly(dimethyl)siloxane polymer preferably has a viscosity around 100-1000 mPas, more preferably about 350 mPas. In an embodiment, the poly(dimethyl)siloxane polymer is dimethicone. The poly(dimethyl)siloxane polymer enhances a soft feel of the wipe as it enhances a lubricant effect, and attributes to protecting a skin after cleaning by forming a stable film on said skin.

In exemplary embodiments, the composition may additionally comprise an emulsifying compound. The emulsifying compound supports to form an emulsion in the composition containing water. In particular, the emulsifying compound may form an emulsion for containing the poly(dimethyl)siloxane polymer. The emulsifying compound comprises at least one component of the group consisting of fatty acid derivate of triglyceride, such as caprylic triglyceride and capric triglyceride, a glucoside, such as lauryl glucoside, a lactylate, such as sodium lauroyl lactylate and sodium stearoyl lactylate, a monoglyceride, such as glyceryl stearate, glyceryl citrate, glyceryl lactate, glyceryl linoleate, glyceryl oleate, and glyceryl caprylate, a fatty alcohol, such as cetearyl alcohol, a fatty acid derivate of a polyglycerol, such as polyglyceryl-4-cocoate and polyglyceryl-3-caprate, and mixtures thereof. The emulsifying compound may suitably be selected such that the components thereof are biodegradable.

In an exemplary embodiment, the composition additionally comprises a preservative compound. The preservative compound prevents decomposition of the composition by microbial growth or by undesirable chemical changes. The preservative compound may be selected from the approved preservative compounds listed in Annex V of Regulation (EC) No 1223/2009 of the European Parliament, entitled the Cosmetic Product Regulation (CPR), online available at: https://eur-lex.europa.eu/legal-content/EN/ALL/?uri=CELEX%3A32009R1223.

In more preferred embodiments, the preservative compound is selected from at least one of a sorbic acid, a derivative of sorbic acid, benzoic acid, a derivative of benzoic acid, lactic acid, propionic acid, one or more parabens, and phenoxyethanol. The preservative compound prevents degradation of the composition by bacteria, moulds and fungi.

In another aspect of the present invention a process is provided for preparing an article comprising the steps:

a. providing i. a textile carrier; ii. a composition according to the present invention; and
b. impregnating the textile carrier by the composition at a temperature ranging from 5° C. to 50° C.

The advantages of the present invention become more apparent in case the impregnating step of the textile carrier by the composition may be carried out at a relatively low temperature. The composition may easily be applied into the textile carrier by impregnation step due to the shear thinning behavior of the composition. In particular, the impregnating step is carried out at a temperature of 5° C. to 50° C., preferably at a temperature of 10° C. to 40° C., more preferably at a temperature of 15° C. to 30° C.

Due to the desirable rheological properties of the composition, applying pressure to the composition during impregnation of the textile carrier with the composition may not be needed. A preferred embodiment of the invention therefore provides a process wherein step b. is carried out substantially pressureless, preferably by spraying the composition onto the textile carrier. This embodiment provides advantages in terms of process efficiency.

The textile carrier (component i.) may be a woven fabric, or may be a non-woven fabric. The textile carrier may comprise a fabric of natural fibers and/or synthetic fibers. A woven fabric may be made by weaving together fibers of silk, cotton, polyester, wool, and similar materials to form an interlocking matrix of loops. Non-woven fabrics, on the other hand, are made by a process that presses a single sheet of material from a mass of separate fibers. Fibers, such as cotton, cellulose and regenerated cellulose, may be used in this process, as well as plastic resins like polyester, polyethylene, and polypropylene, or mixtures thereof. The textile carrier may be presented as a sheet or wipe, but other geometries may be applicable, such as a glove to cover a hand, or a cap to cover a user's hair. Corresponding articles then have about the same geometry. The loading may be adapted accordingly, wherein the loading represents the weight ratio of the textile carrier relative to the composition.

The composition (component ii.) may be any composition according to the present invention. The composition (component ii.) may be suitably selected for impregnating the textile carrier (component i.).

In an exemplary embodiment, in step a. the composition ii. is provided inside a container, the process additionally comprising the step: c. pumping the composition from the container towards the textile carrier at a temperature of 5° C. to 50° C. During the pumping process the composition is easily transported, such as through supply tubes, towards the textile carrier due to its shear thinning behavior. In particular, the pumping step is carried out at a temperature of 5° C. to 50° C., preferably at a temperature of 10° C. to 40° C., more preferably at a temperature of 15° C. to 30° C.

In another aspect of the present invention an article, preferably a wipe, is provided, comprising a textile carrier and the composition according to the present invention.

In another aspect of the present invention a use is provided of the article according to the present invention for a topical application of the composition onto a skin. The article including the composition according to the present invention supports to apply a relatively abundant layer of the composition onto a skin when wiping the article over the skin. The relatively abundant layer of the composition attributes and/or enhances a moisturizing effect of the skin and supports a protection of the skin.

### EXAMPLES

The following examples further illustrate the invention but are not to be construed as limiting its scope. In the following, all amounts given in the Tables are parts by weight if not indicated otherwise.

### Raw Materials

| | |
|---|---|
| Aq | Water |
| SMEC 1 | Veegum Ultra, Bentonite clay |
| | (manufacturer Vanderbilt Minerals LLC) |
| ACRPOL 1 | Aristoflex Velvet, acrylamidepolymer |
| | (manufacturer Clariant International Ltd) |
| PR-GL | Propylene glycol |
| GLY | Glycerin |
| HYAL 1 | Hyaluronic acid, sodium salt of |
| DIMET | Dimethicone |
| EMUL 1 | Emulsogen CCT |
| | (manufacturer Clariant International Ltd) |
| PRES | Suitable preservative mix, including e.g. |
| | sorbic acid, potassium sorbate |

The SMEC 1, Veegum Ultra, consists of magnesium aluminum silicate, preferably variants with fast solubility in water. The DIMET dimethicone has a viscosity of 100-1000 cP, and preferably 350 cP. The EMUL 1, Emulsogen CCT, contains a mixture of Caprylic/Capric Triglyceride (and) Aqua (and) Glycerin (and) Laureth-23 (and) Behenyl Alcohol (and) Disodium Ethylene Dicocamide (and) Glyceryl Stearate (and) Sodium Lauroyl Lactylate (and) Glyceryl Stearate Citrate.

### Nonwoven Materials

Wet wipes and associated nonwoven products are designed to release lotions when used, and to absorb and retain during the manufacturing process and upon storage. The used lotions are mostly water based. The nonwoven materials used must therefore contribute to an adequate balance of hydrophobic and hydrophilic properties, thus ensuring good impregnation, product stability, and product functionality when used.

The nonwoven material consists of natural or man-made polymers, or a mixture thereof. Typical natural materials would include cotton, wood pulp, cellulose, silk and wool, typical man-made materials include nylon, polyester, polyethylene, polypropylene, polylactic acid, regenerated cellulose and acrylics. The nonwoven material is preferably a blend of polyester first fibers and regenerated cellulose second fibers. Preferably, the regenerated cellulose is lyocell. The fibers are blended by mechanical entanglement, such as spun lacing or needle punching. Preferably, the hydrophobic polyester fibers constitute at least 40% of the blend by weight, while the hydrophilic fibers constitute at most 60% of the blend by weight. The nonwoven material may or may not be cross-lapped.

### Measurement Methods

Viscosity. Yield Point and Thixotropy

When the dynamic viscosity of a composition is too high to enable pumping it through a piping system, an alternative would be to select or create a composition that becomes sufficiently fluid when force is applied.

The yield point (τ0, also called yield stress) is the lowest shear-stress value above which a material will behave like a fluid, and below which the material will act like a solid. The yield point is the minimum force that must be applied to a material so that it starts to flow. If the yield stress is too high, the composition will not flow under desired manufacturing conditions, and will remain solid. If the yield stress is too low, flow will occur too easily. This would be interesting for pumping and impregnating, but the impregnated nonwoven product will be unstable.

Thixotropy (Δ) may be defined as the continuous decrease of apparent viscosity with time under shear and the subsequent recovery of the viscosity when the flow is discontinued. Thixotropic nature of a fluid is characterized by a hysteresis curve. The surface area of the hysteresis curve is linear proportional to the magnitude of thixotropy. A small surface area means low thixotropy, and denotes rapid return of the composition to its solid state. The thixotropy may be too low to properly impregnate the nonwoven material. A large surface area means high thixotropy, and denotes slow return of the composition to its solid state. The composition remains too runny for too long, resulting in suboptimal dispersion of the composition in the nonwoven cloth.

Measuring thixotropy (Δ), yield stress (τ0) and viscosity (η) requires a rheometer. A typical rheometer, used for characterizing this invention, is the Haake Viscotester IQ of Thermo Fisher Scientific Inc., which is a rotational rheometer equipped with a coaxial cylinder geometry (for instance the CC25 DIN/Ti geometry). The geometry operates according to ISO standard 3219.

The viscosity, the yield point and the thixotropy of a composition are measured according to ISO 3219. The measurement is carried out at 23.0° C. The viscosity is determined at a shear rate of 1/100 s⁻¹ over 60 s.

The yield point is determined after the composition has had sufficient time to recover any thixotropic structure. The yield point is expressed in [Pa] and determined at a constant shear rate of at least 1/10000 s⁻¹ as a logarithmic shear stress curve of 0.001-500 Pa over 60 s.

The thixotropy is determined quantitatively after the composition has had sufficient time to recover any thixotropic structure by determining the shear stress vs. shear rate hysteresis loop, at a shear rate of 1/10000−1000 s⁻¹ over 60 s (linear, forward curve), 1000 s⁻¹ over 30 s, and 1000-1/10000 s⁻¹ over 60 s (linear, backward curve). The thixotropy is expressed in [Pa/s] and calculated as the difference in area under the curve between the forward curve and the backward curve.

### Application and Performance

### Preparation of the Composition

The preparation of the composition is separated into several steps for better clarity. The steps are:
1. Provide at least 75 weight-% of the water
2. Add the layered silicate component and the acrylpolymer in the water
3. Mix the mixture of step 2. to form a stable suspension of the layered silicate component and the acrylpolymer in water
4. Optionally add preservatives to the mixture of step 3.
5. Optionally prepare a premixture of the dimethicone and the emulsifier compound in water, and add the premixture of the dimethicone and the emulsifier compound in water to the mixture of step 4 or step 3.
6. Add the humectant, optionally including other ingredients, such as a hyaluronic acid component, to the mixture of step 3, 4, or 5.
7. Add water to the mixture such that the total amount of water is supplied.

The following compositions were prepared according to the steps explained above:

TABLE 1
compositions

| | Example 1 | Example 2 | Example 3 | Example 4 | Example 5 | Example 6 | Example 7 |
|---|---|---|---|---|---|---|---|
| Ingredient | [wt %] | [wt %] | [wt %] | [wt %] | [wt %] | [wt %] | [wt %] |
| SMEC 1 | 1.30 | 1.30 | 1.00 | — | 1.30 | 1.30 | 1.30 |
| ACRPOL 1 | 0.40 | 0.40 | 0.40 | 0.40 | 0.30 | 0.50 | — |
| PR-GL | 2.00 | 2.00 | 2.00 | 2.00 | 2.00 | 2.00 | 2.00 |
| GLY | 1.00 | 1.00 | 1.00 | 1.00 | 1.00 | 1.00 | 0.50 |
| HYAL 1 | — | 0.05 | 0.05 | 0.05 | 0.05 | 0.05 | 0.05 |
| DIMET | 3.00 | 3.00 | 3.00 | 3.00 | 3.00 | 3.00 | 3.00 |
| EMUL 1 | 3.00 | 3.00 | 3.00 | 3.00 | 3.00 | 3.00 | 3.00 |
| PRES | 0.70 | 0.70 | 0.70 | 0.70 | 0.70 | 0.70 | 0.70 |
| Aq* | Qs | Qs | Qs | Qs | Qs | Qs | Qs |
| Total | 100.00 | 100.00 | 100.00 | 100.00 | 100.00 | 100.00 | 100.00 |

Qs: the amount of water (Aq) is such that the total of the ingredients is 100.00 wt %. The total weight of the suspension system (the layered silicate and the acrylpolymer) and the weight ratio between the layered silicate and the acrylpolymer for the Examples 1-7 is shown in the following Table:

TABLE 2
suspension system [wt %] and weight ratio [/]

| Ingredient | Example 1 | Example 2 | Example 3 | Example 4 | Example 5 | Example 6 | Example 7 |
|---|---|---|---|---|---|---|---|
| suspension | 1.70 | 1.70 | 1.40 | 0.40 | 1.60 | 1.80 | 1.30 |
| system [wt. %] | | | | | | | |
| Weight Ratio [/] | 3.25 | 3.25 | 2.50 | 0.00 | 4.33 | 2.60 | >> |

### Preparation of the Wipe

The preparation of the wipe is separated into several steps for better clarity. The steps are:
1. Provide a textile carrier
2. Provide a composition according any one of the examples in a container
3. Pump the composition towards the textile carrier at a temperature of about 10-30° C.
4. Apply the composition onto the textile carrier, while shearing the composition
5. Let the composition impregnate the textile carrier at a temperature of about 10-30° C.

The textile carrier used in these tests is a non-woven fabric, such as a fabric containing cotton fibers.

Each of the compositions according to the examples 1-6 is used to impregnate a non-woven fabric. Each of the compositions shows a shear thinning behavior, which makes them suitable to pump the composition towards the textile carrier at a temperature of about 15-30° C. and to impregnate the textile carrier at a temperature of about 15-30° C.

Additionally, each of the compositions is sufficiently thick at a temperature of about 15-30° C. to provide a sufficient abundant layer on a skin, when wiping the wipe including the composition over the skin.

Each of the wipes having a composition according to the examples 1-6 is evaluated on skin feel sensation, when wiping the wipe over the skin. A skin feel is a sense of softness of the wipe when manually wiping the wipe over the skin. The wiping test was performed at ambient temperature of about 15-30° C.

Examples 1, 2 and 6 showed an appropriate skin feel. Examples 3-5 showed a reasonable, but lesser, skin feel. It is assumed that the weight ratio between the layered silicate and the acrylpolymer is relevant for the skin feel of the wipe. In these examples, a higher weight ratio of higher than 2.5 correlates to a more thickened composition at a temperature of about 15-30° C.

The viscosity, yield point and thixotropy index are shown in Table 3.

TABLE 3
viscosity, yield point and thixotropy index.

| | Example 1 | Example 2 | Example 3 | Example 4 | Example 5 | Example 6 | Example 7 |
|---|---|---|---|---|---|---|---|
| Viscosity (η) (mPas) | 124 | 178 | 159 | 155 | 113 | 226 | unstable |
| Yield Point (τ0) (Pa) | 74 | 115 | 110 | 114 | 48 | 22 | unstable |
| Thixotropic (Δ) (Pa/s) | 500 | 383 | 2418 | 1943 | 0 | 2310 | unstable |

The results show that the layered silicate, preferably the smectite, plays a role in reducing the thixotropy. A composition containing less (Example 3) or no (Example 4) layered silicate has a thixotropy which is too high for this type of application. The composition stays “runny” for too long, resulting in the composition not sticking to but going through the textile upon impregnation. Additionally, when the composition has less (Example 5) or no (Example 7) acrylate polymer, the composition either does not have a thixotropy behaviour (see Example 5) or does not have a stable viscosity, yield point and thixotropy (Example 7). Both are unsuitable for application in impregnated textiles at room temperature. Example 6 shows that too much acrylate polymer disturbs the balance with the layered silicate, resulting in increased dynamic viscosity combined with a thixotropy which is too high for this type of impregnation application. Addition of Sodium Hyaluronate (Example 2) further optimises dynamic viscosity, without negatively affecting the thixotropy. Additionally, the thixotropic index increases when the weight ratio is close to 2.60 and lower or the weight ratio is at least 4.30. The thixotropic index is preferably controlled to be in the range 100 to 1000 Pa/s, preferably between 250 and 750 Pa/s.

Whilst the principles of the invention have been set out above in connection with specific embodiments, it is to be understood that this description is merely made by way of example and not as a limitation of the scope of protection which is determined by the appended claims.

1. A composition comprising at least a layered silicate, an acrylate polymer, a preservative compound, a humectant compound and water, wherein the composition further comprises hyaluronic acid or a salt derivative thereof.

2. The composition according to claim 1, wherein the amount of the hyaluronic acid or the salt derivative thereof is up to 0.5% wt. % based on the total weight of the composition.

3. The composition according to claim 1, wherein the molecular weight of the hyaluronic acid or the salt derivative thereof is at least 800 kDa, and more preferably at least 1500 kDa.

4. The composition according to claim 1, wherein the salt derivative of the hyaluronic acid is selected from at least one of a sodium salt, a potassium salt, and a zinc salt.

5. The composition according to claim 1, wherein the layered silicate is a smectite, preferably selected from the group consisting of bentonite, hectorite, montmorillonite, nontronite, beidellite, saponite, stevensite and mixtures thereof, more preferably a bentonite.

6. The composition according to claim 1, wherein the amount of the layered silicate is at least 0.3 wt-%, preferably at least 0.5 wt-%, more preferably at least 0.8 wt-%, based on the total weight of the composition.

7. The composition according to claim 1, wherein the acrylate polymer comprises acrylamide units, preferably a part of the acrylamide units comprising a sulphonic acid group, which is modified by an ammonium salt.

8. The composition according to claim 1, wherein the amount of the acrylate polymer is at most 1.0 wt-%, preferably at most 0.5 wt-%, based on the total weight of the composition.

9. The composition according to claim 1, wherein the weight ratio between the layered silicate and the acrylate polymer is in the range from 1.0:1.0 to 5.0:1.0, preferably from 2.6:1.0 to 4.3:1.0.

10. The composition according to claim 1, having a dynamic viscosity according to ISO 3219 at a temperature of 23° C. exceeding 50 mPas, preferably exceeding 100 mPas.

11. The composition according to claim 1, having a yield point according to ISO 3219 at a temperature of 23° C. between 25 and 250 Pa, preferably between 50 and 150 Pa.

12. The composition according to claim 1, having a thixotropy according to ISO 3219 at a temperature of 23° C. of 100 to 1000 Pa/s, preferably between 250 and 750 Pa/s.

13. The composition according to claim 1, wherein the humectant compound comprises at least one of an alkyl glycol, a glycerin, and a glycerin derivative.

14. The composition according to claim 1, further comprising a poly(dimethyl)siloxane polymer.

15. The composition according claim 1, further comprising an emulsifying compound, wherein the emulsifying compound comprises at least one component of the group consisting of fatty acid derivate of triglyceride, a glucoside, a lactylate, a monoglyceride, a fatty alcohol, a fatty acid derivate of a polyglycerol, and mixtures thereof.

16. An article for personal care, comprising a textile carrier and a composition according to claim 1.

17. Use of the article according to claim 16 for at least one of skin moisturisation or hydration, skin regeneration, restoration of skin barrier, relief or prevention of skin blemishes, treating age related skin conditions, relief or prevention of skin infections, skin wound healing, relief or prevention of dermatitis, or for enhancing anti-acne properties of the skin.

18. Process for preparing an article for personal care, comprising the steps of:
a. providing
i. a textile carrier;
ii. a composition according to claim 1;
and
b. impregnating the textile carrier with the composition at a temperature ranging from 5° C. to 50° C.

19. Process according to claim 18, wherein step b. is carried out substantially pressureless, preferably by spraying the composition onto the textile carrier.
